(19) United States
(12) Patent Application Publication
(10) Pub. No.: US 2007/0010855 A1
(43) Pub. Date: Jan. 11, 2007

(54) Facial lifting needle and method thereof

(76) Inventor: Maximiliano Eduardo Florez Mendez, Lima (PE)

Correspondence Address: DEFILLO & ASSOCIATES, INC., 4922 EAGLE COVE SOUTH DRIVE, PALM HARBOR, FL, 34685, US

(21) Appl. No.: 11/175,054

(22) Filed: Jul. 5, 2005

(51) Int. Cl. A61B 17/06

(52) U.S. Cl. 606/222

(57) ABSTRACT

The present invention concerns a facial lifting needle having a horizontal solid surgical steel needle with two tips at each end of the needle. Both tips have single bevel (one bevel). An opening is located at 6 mm distal from one of the tips. The opening is configured to deliver surgical thread into the tissue. The present invention also concerns with a method of lifting facial tissue of the skin.

### TECHNOLOGICAL FIELD

The present invention concerns a device for facial lifting.

### DESCRIPTION OF THE PRIOR ART

When performing a facial lifting, the current device comprises a needle with only one tip or point and an opening at the distal end of the needle for delivering the surgical thread. The device penetrates the skin in the region to treat delivering only one thread of the surgical thread, reaches the subcutaneous tissue, then it is pushed through to emerge through the skin at a selected emerging point. The needle is totally pulled out of the skin at the selected emerging point located in the distal edge of the region to treat (FIGS. 1 and 2, the device is used in an eyebrow tail region).

Then the needle is pushed again into said selected emerging point in the skin delivering the second thread of the surgical thread located at the distal end of the needle, through the subcutaneous tissue in order to enable the traction and facial lifting. The needle is ejected through said penetrating point.

The traction of both threads of the surgical thread enables to perform the facial lifting of the treated region. At this moment, a pinch can be seen in the region to be lifted. The pinch is the result of the surgical thread being located subcutaneously except in the needle emerging region where it comes to the surface to begin the second penetration back to the penetrating point delivering the second thread of the surgical thread (FIGS. 3 and 4).

The problem that the invention intends to solve it is avoiding the coming to surface of the needle allowing the surgical thread to stay subcutaneously avoiding the pinches.

### DESCRIPTION OF THE DRAWINGS

FIGS. 1-4 show how the needle of the present invention is used in an eyebrow tail region.

FIG. 5 shows the needle of the present invention.

FIG. 5a shows an enlargement of the tip of the needle according to the present invention.

FIG. 6 shows the position of the needle in respect to the penetrating point of the incision.

FIG. 7 shows how the surgical thread emerges from the subcutaneous tissue and through the skin.

FIG. 8 shows how the needle is pushed for enabling the delivery of the surgical thread so that the thread reaches a selected internal anchoring point.

FIG. 9 shows how the needle is rotated for instance 45° at the selected internal anchoring point.

FIG. 10 shows how the thread is pushed through the emerging point into the tissue addressing it to the penetrating point in the incision.

FIG. 11 shows how the needle is totally out of the skin, the thread is prepared to traction and lift the region to be treated.

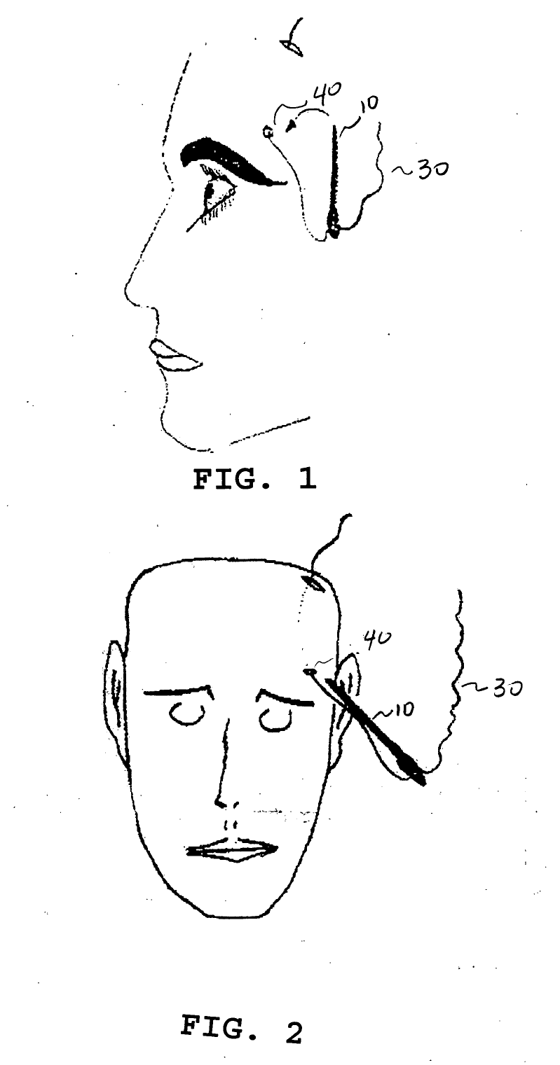

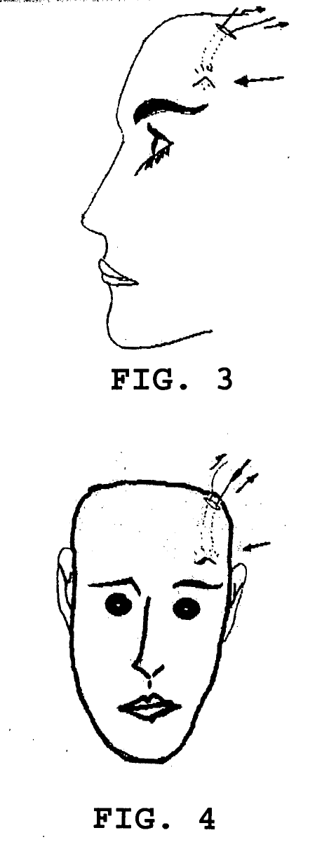

### DETAILED DESCRIPTION OF THE INVENTION

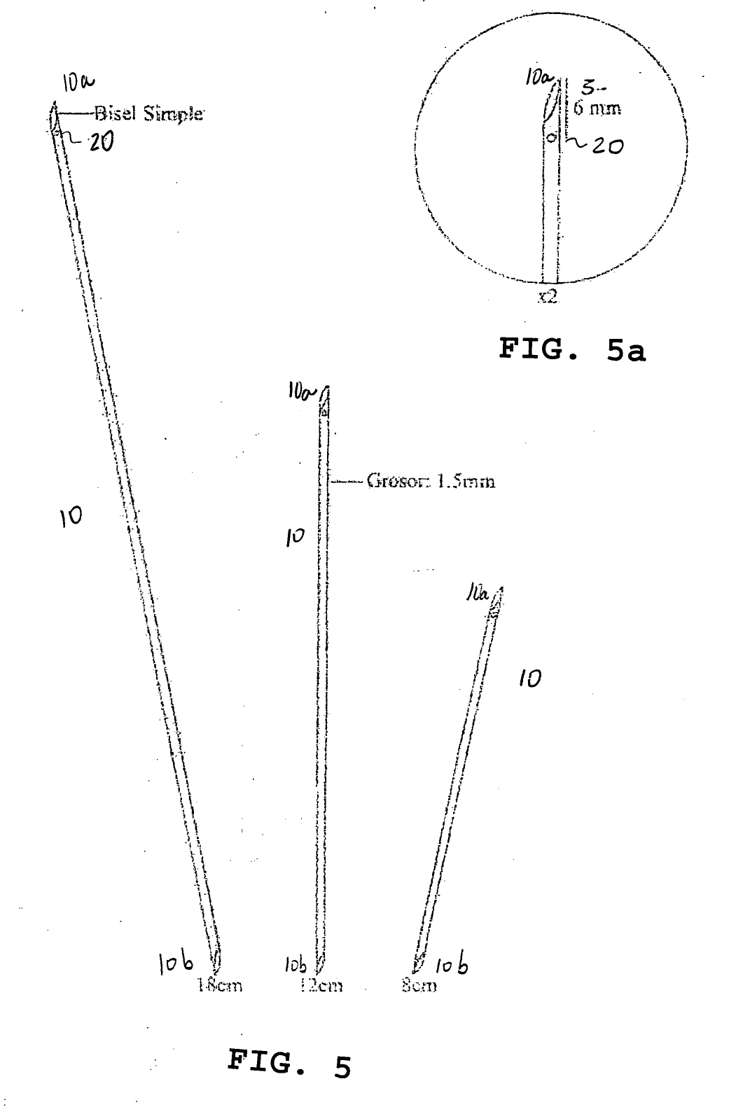

The invention comprises a horizontal solid surgical steel needle 10 with two tips (10a, 10b), one at each end of the needle. Both tips have simple bevel (one bevel). An opening 20 is located 6 mm distal from one of the tips. Said opening is configured to deliver surgical thread 30 into the tissue. The diameter of said needle is 1.5 mm, and the length can be 8 cm, 12 cm, 18 cm. (FIG. 5).

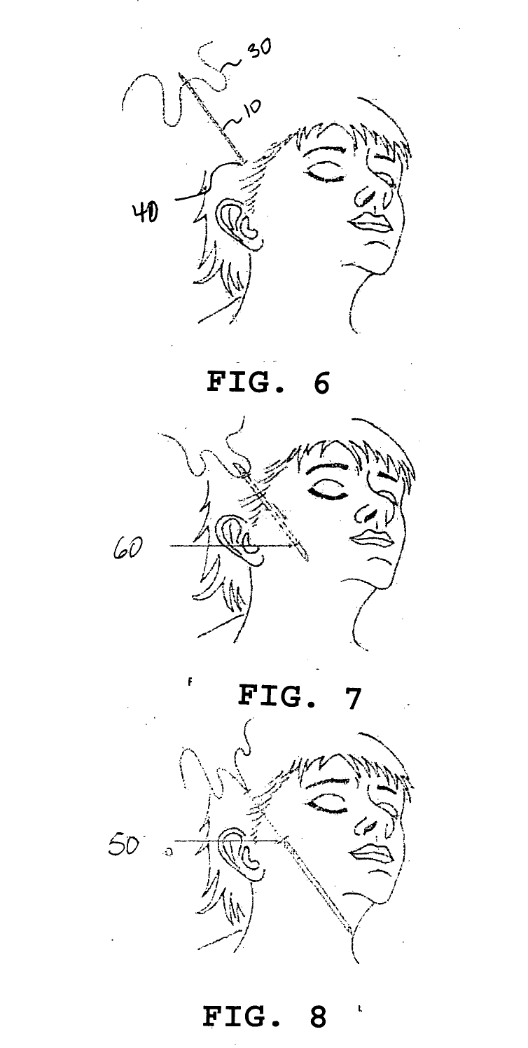

An incision 40 of 1 cm is performed in a selected region of the scalp. A surgical thread 30 is threaded through the opening 40. The needle 10 penetrates the skin and the subcutaneous tissue with the tip distal from the tip that delivers the surgical thread through said incision (FIG. 6).

The tip distal from the tip that delivers the surgical thread emerges from the subcutaneous tissue at an emerging point 60 and through the skin to 2 cm from the internal anchoring point (FIG. 7).

The needle is pushed in order to emerge approximately ¾ of its length enabling the delivery of the surgical thread so that the thread reaches a selected internal anchoring point 50 (FIG. 8).

The needle is rotated for instance 45° at the selected internal anchoring point 50 (FIG. 9). This rotation enables to perform a curl with the surgical thread.

Then the tip delivering the thread is pushed through the emerging point into the tissue addressing it to the penetrating point in the incision. (FIG. 10).

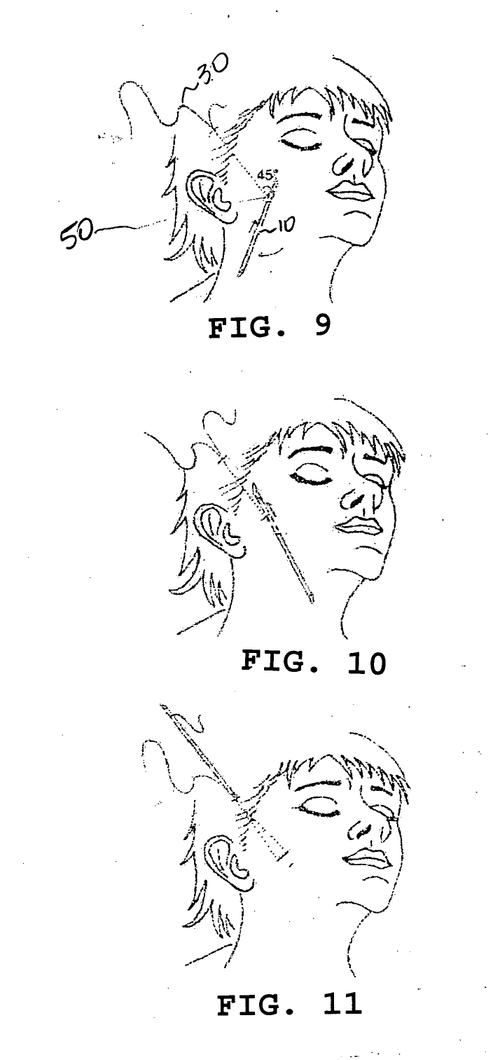

Once the needle is totally out of the skin, the thread is prepared to traction and lift the region to be treated (FIG. 11).

### DESCRIPTION OF THE INVENTION

The needle penetrates the subcutaneous tissue with the tip distal to the surgical thread. Said tip emerges from the subcutaneous tissue and goes through the skin at a certain distance from the penetrating point. At the emerging point, the needle is extracted approximately ¾ of its length. This fact enables that the surgical thread is located at the selected anchoring point inside the subcutaneous tissue.

At said anchoring point a rotation is done inside the subcutaneous tissue so that the surgical thread makes a curl or curve. Then the needle is pushed back parallel to the penetrating way to emerge through the penetrating point at the incision.

This way the thread went through the same path as the needle, remaining in the subcutaneous tissue by a distal end, and both threads out of the tissue by the penetrating point. Both threads of the surgical thread are tractioned so that they lift the facial tissue (skin and lipid tissue).

The prior art is a needle that penetrates but should be totally ejected from the facial tissue to do the parallel return by its beveled tip changing the plane causing pinches and retractions in the skin and subcutaneous tissue.

The needle penetrates the subcutaneous tissue with the tip distal to the surgical thread. Said tip emerges from the subcutaneous tissue to the skin at a certain distance of the penetrating point. At this moment, the needle is pushed out of the subcutaneous tissue and skin approximately ¾ of its length so that the surgical thread remains into the subcutaneous tissue because said thread penetrates the subcutaneous tissue as if dragging. At this moment, a rotation is done inside the tissue so that the surgical thread is curved or curled. The needle goes back in a parallel path in relation to the penetrating path to emerge through the penetrating point in the incision. The tip delivering the surgical thread emerges first. This way the surgical thread goes through an identical path from the one that the needle does, one of its ends remains inside the subcutaneous tissue making a curl or curve and both threads of the surgical thread emerge out of the tissue through the penetrating point in the incision. When both threads of the surgical thread are tractioned, the lifting of the facial tissue is done.

As an example of use, this needle has been successfully used when lifting eyebrows tail, temples, and cheeks. It could be used in other regions of the human body.

What is claimed is:

1. A facial lifting needle comprising:
a solid surgical steel needle having a first end and a second end, two simple beveled tips one at each end of the needle; and an opening near the first beveled tip.

2. The facial lifting needle according to claim 1, wherein the opening is located at 6 mm from the first beveled tip.

3. The facial lifting needle according to claim 1, wherein the needle has a diameter of 1.5 mm.

4. The facial lifting needle according to claim 1, wherein the needle has a length of 8 cm, 12 cm, or 18 cm.

5. A method of lifting facial tissue of the skin comprising:
a) providing a facial lifting needle comprising a solid surgical steel needle having a first end and a second end, two simple beveled tips one at each end of the needle; and an opening near the first beveled tip, wherein the opening is adapted to carry a surgical thread;
b) penetrating the second tip of the needle through a subcutaneous tissue at an incision opening;
c) emerging from the subcutaneous tissue to the skin with the second tip at a distance from the incision opening;
d) pulling the needle out of the skin approximately ¾ of its length;
e) rotating the needle so that the surgical thread is curved or curled at a selected internal anchoring point inside the subcutaneous tissue;
f) introducing the needle into the subcutaneous tissue by the first tip so that the threads goes back in a parallel path in relation to the one did by the needle;
g) extracting the needle by the incision opening; and
h) tractioning both threads of the surgical thread in order to lift the facial tissue.
